(19) United States
(12) Patent Application Publication
(10) Pub. No.: US 2005/0101607 A1
(43) Pub. Date: May 12, 2005

(54) Pharmaceutical composition containing a beta-3-adrenoceptor agonist and an alpha antagonist and/or a 5-alpha reductase inhibitor

(75) Inventors: Martin Michel, Amsterdam (NL); Ursula Ebinger, Boonton Township, NJ (US); Marion Wienrich, Weiterstadt (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/980,665

(22) Filed: Nov. 3, 2004

(30) Foreign Application Priority Data
Nov. 3, 2003 (DE) 103 51 539
Nov. 4, 2003 (DE) 103 52 131

(51) Int. Cl. A61K 31/517; A61K 31/506; A61K 31/4545

(52) U.S. Cl. 514/252.14; 514/318; 514/252.16; 514/266.21

(57) ABSTRACT

This invention describes a new combination for the treatment of functional bladder disorders which comprises an alpha antagonist and/or 5-alpha-reductase inhibitor and a beta-3-adrenoceptor agonist.

This invention relates to a new active substance combination for the treatment of complaints connected with morbid changes to or irritation of the prostate in a mammal and possibly associated with functional bladder disorders. The invention proposes a pharmaceutical active substance combination of at least one beta-3-adrenoceptor agonist and at least one other active substance which is selected from among the alpha antagonists (alpha adrenoceptor antagonists) and/or the 5-alpha-reductase inhibitors. Preferably, the combination according to the invention is used in patients in whom there is both a functional bladder disorder and morbid changes to or irritation of the prostate. The functional bladder disorders may give rise to urinary incontinence.

### PRIOR ART

The incidence of diseases of the urogenital tract is constantly increasing as a result of a shift in the ageing statistics. Apart from the medical consequences such as chronic infections of the urinary passages, the symptoms of the disease are associated with a high psychological burden of suffering.

The lower urinary tract consists of the bladder, the urethra, the corresponding muscles, and the ligaments of the suspensory apparatus. In men, the prostate is also part of this tract.

The purpose of the bladder is to store the urine and evacuate it. The important factors for performing the storage function are not only the relaxation of the bladder muscle (detrusor muscle), but also the closure mechanisms provided by the neck of the bladder and the smooth muscle of the urethra, and also by the cross-striated muscle of the urethra and the pelvic floor. During the emptying of the bladder (micturition), the detrusor muscle contracts while the urethra and pelvic floor relax and the sphincter muscle of the bladder opens. These processes require complex control by the parasympathetic, sympathetic, and somatic nervous system.

The prostate, whose functions include, inter alia, forming secretions for the spermatic fluid, is located between the bladder and the part of the pelvic floor which forms the outer sphincter muscle of the urethra. The prostate surrounds the start of the urethra. The muscles of the prostate and bladder functionally engage with one another to some extent, e.g., when passing water, when the ejaculatory ducts and the prostatic ductules are closed off by the muscles of the bladder and prostate so that no urine can enter. Conversely, the prostate supports the neck of the bladder and hence helps to close off the bladder.

In men with prostate problems, irritations often occur when passing water, particularly when there is benign prostatic hyperplasia (BPH). This disease occurs in more than 50% of men over 50 and leads to enlargement of the prostate. The reason for it is an increase in the number of cells, although they remain the same size. As a result of the enlargement of the prostate the urethra may be constricted in this region and total emptying of the urethra may be delayed. The irritable symptoms may be intensified both during the filling phase of the bladder and during emptying of the bladder. If there is also a functional bladder disorder, the irritable symptoms are increased still further.

It is also easier for a chronic urinary tract infection to develop when there is benign prostatic hyperplasia combined with a functional bladder weakness.

Other forms of prostate disease or irritation which may interact with bladder function are various forms of prostatitis. The term prostatitis embraces a heterogeneous clinical picture with multiple causes. A distinction is drawn, on the one hand, between acute and chronic, mostly non-specific inflammation or irritation of the prostate and, on the other hand, between bacterial or antibacterial causes. The two phenomena may both be present. Some forms of prostatitis are also known as non-inflammatory chronic pain syndrome of the pelvis, pelvic myoneuropathy, prostatodynia, or prostatopathy.

Alpha antagonists or alpha reductase inhibitors are used to treat functional symptoms of benign prostatic hyperplasia (BPH).

Alpha antagonists can bind selectively and competitively to the postsynaptic alpha-1 receptors, particularly to subtypes alpha1A and alpha1D. As a result, the smooth muscle of the prostate and urethra is relaxed and the tone of the smooth muscle of the prostate and urethra is reduced. Consequently the flow of urine is increased. 5-alpha-reductase inhibitors inhibit the enzyme 5-alpha-reductase. This enzyme converts the body's testosterone into dihydrotestosterone, which directly stimulates the growth of the prostate tissue.

Prostate problems may be made worse by the occurrence of functional bladder disorders and vice versa.

Functional bladder problems are a heterogeneous group of disorders which differ in their aetiology, diagnosis, and therapy. In the standardizing recommendations of the International Continence Society (ICS), urinary incontinence is defined as involuntary loss of urine, which is objectively detectable and constitutes a social and hygiene problem. Generally, urinary incontinence only occurs when there is an unintentional increase in the pressure in the bladder during the storage phase. This can happen as a result of unrestricted contractions of the detrusor muscle (urge incontinence) or failure of the urethral closure mechanism (stress incontinence).

According to the ICS definition, overactive bladder (OAB) is characterized by an irresistible imperative need to urinate, which may or may not be associated with urge incontinence, usually with increased frequency of micturition and nocturnal urination. Pathophysiologically, this complaint may be based on involuntary contractions during the filling phase, the cause of which may be neurogenic or non-neurogenic (idiopathic) in nature.

Urge incontinence is characterized by an irresistible urge to urinate and involuntary loss of urine.

Stress incontinence is characterized by the involuntary loss of urine which generally occurs at moments of elevated intraabdominal pressure. This may occur for example when lifting, coughing, sneezing, or running, while at the same time there is no detrusor activity. Loss of urine takes place as the result of a variable combination of an insufficiency of the sphincter muscles of the bladder and the pelvic floor, as well as anatomical defects in the suspensory apparatus. As a result the closure pressure of the urethra is too low and incontinence results. In men, this form of urinary incontinence is usually only observed after prostatectomies or other surgical interventions on the small pelvis.

In so-called mixed incontinence, patients suffer from symptoms of both stress incontinence and urge incontinence. Once again, it is mainly women who are affected. For treating the various forms of functional bladder disorders, particularly stress incontinence, urge incontinence, mixed incontinence or overactive bladder, various therapeutic approaches are available.

For treating urge incontinence, the WHO recommends treatment with anticholinergics (antimuscarinics). However, their use is limited because they are only moderately effective and particularly because they have serious side effects such as dryness of the mouth, accommodation disorders, constipation, and central nervous effects (dizziness, fatigue, an dconfusion).

Stress incontinence is treated primarily by conservative and surgical procedures. Up till now there has been no generally suitable drug therapy available. Alpha-agonists such as pseudoephedrine and phenylpropanolamine have shown some effect, albeit very modest, in the treatment of low-grade stress incontinence. A disadvantage is that they have no selectivity for the urethral muscles and have numerous side effects such as hypertension, tachycardia, arrhythmia, sleep disorders, headaches, and tremors.

The treatment of mixed incontinence is a controversial subject of discussion and comprises combinations of invasive procedures for treating the stress incontinence component and drug therapies for treating the urge incontinence component.

Since the mid-1995's, it has been reported that selective beta-3-adrenoceptor-agonists are also promising in the treatment of urinary incontinence (EP 0 958 835). As the stimulation of beta-3-receptors is of exceptional importance for the relaxation of the detrusor muscle, the use of selective beta-3-adrenoceptors in patients with urge incontinence should result in the reduction or prevention of involuntary detrusor contractions during the urine storage phase. Tests with beta-3-adrenoceptor agonists indicate that they will be highly effective while being well tolerated. In addition, their activity should be restricted to the storage phase of the bladder and unimpeded emptying of the bladder should be guaranteed without any build-up of urine residues.

### SUMMARY OF THE INVENTION

Despite new approaches both in the treatment of prostate problems and in the control of bladder function, the development of efficient and well-tolerated therapies remains a challenge. This is particularly true when there are prostate problems, such as benign prostatic hyperplasia or prostatitis, and particularly when there are also functional bladder disorders at the same time.

The present invention sets out to contribute to the improved treatment of the affected patients.

One aim of the invention is therefore to provide medication for patients with prostate problems, particularly benign prostatic hyperplasia or prostatitis.

A further aim is to provide medication for patients with prostate problems, which contributes to improved control of bladder function.

Another aim relates to a medication for the treatment of benign prostatic hyperplasia and/or prostatitis when there is also a functional bladder disorder such as, for example, stress incontinence, urge incontinence, mixed incontinence, or overactive bladder with or without urge incontinence.

For this, a pharmaceutical composition is proposed, which is intended to combine the advantages of the alpha antagonists for treating benign prostatic hyperplasia or prostatitis and those of the beta-3-adrenoceptor agonists for controlling bladder function in a manner which is favorable to the associated irritations.

### DESCRIPTION OF THE INVENTION

According to the present invention a new pharmaceutical composition is provided, which contains, as active ingredients, (a) at least one alpha antagonist in a pharmaceutically effective amount and/or a 5-alpha-reductase inhibitor in a pharmaceutically effective amount, and (b) at least one beta-3-adrenoceptor agonist in a pharmaceutically effective amount.

a) Active Components

In the description of the preferred embodiment certain terminology will be used hereinafter in the interests of clarity. This terminology should include the embodiment described and all technical equivalents which work in a similar manner for a similar purpose to achieve similar results. To the extent that any pharmaceutically active compound is disclosed or claimed, it is expressly intended that all active metabolites which are produced in vivo are included, and it is expressly intended that all enantiomers, diastereomers, or tautomers are included, if the compound is capable of occurring in its enantiomeric, diastereomeric, or tautomeric form. Obviously, the isomer which is pharmacologically most effective and most free from side effects is preferred. Also, included are pharmacologically acceptable salts thereof. Examples of pharmaceutically active salts for each of the compounds, which are the subject of this description include, without being restricted thereto, salts, which are prepared from pharmaceutically acceptable acids or bases, including organic and inorganic acids and bases. If the preferred compound is basic, salts may be prepared from pharmaceutically acceptable acids. When selecting the most preferred salt, or to clarify whether a salt or the neutral compound is used, properties, such as bioavailability, ease of manufacture, workability, and shelf life, are taken into consideration, inter alia. Suitable pharmaceutically acceptable acids include acetic acid, benzenesulphonic acid (besylate), benzoic acid, p-bromophenylsulphonic acid, camphorsulphonic acid, carbonic acid, citric acid, ethanesulphonic acid, fumaric acid, gluconic acid, glutamic acid, hydrobromic acid, hydrochloric acid, hydriodic acid, isethionic acid, lactic acid, maleic acid, malic acid, mandelic acid, methanesulphonic acid (mesylate), mucinic acid, nitric acid, oxalic acid, pamoic acid, pantothenic acid, phosphoric acid, succinic acid, sulphuric acid, tartaric acid, p-toluenesulphonic acid and the like. Examples of pharmaceutically acceptable salts include, without being restricted thereto, acetate, benzoate, hydroxybutyrate, bisulphate, bisulphite, bromide, butyne-1,4-dioate, caproate, chloride, chlorobenzoate, citrate, dihydrogen phosphate, dinitrobenzoate, fumarate, glycollate, heptanoate, hexyne-1,6-dioate, hydroxybenzoate, iodide, lactate, maleate, malonate, mandelate, metaphosphate, methanesulphonate, methoxybenzoate, methylbenzoate, monohydrogen phosphate, naphthalene-1-sulphonate, naphthalene-2-sulphonate, oxalate, phenylbutyrate, phenylproprionate, phosphate, phthalate, phenylacetate, propanesulphonate, propiolate, propionate, pyrophosphate, pyrosulphate, sebacate, suberate, succinate, sulphate, sulphite, sulphonate, tartrate, xylenesulphonate, and the like.

Insofar as it is necessary for completeness, the methods of synthesis of the compounds for which the prior art is mentioned and the dosages thereof are expressly included by reference to the prior art mentioned at the corresponding point.

The alpha antagonist is preferably selected from the following group: aa) alfuzosin, ab) bunazosin, ad) doxazosin, ae) indoramin, af) naftopidil, ag) prazosin, ah) tamsulosin, ai) terazosin, aj) urapidil, ak) silodosin (KMD 3213), am) moxisylyte, an) metazosin, ao) fiduxosin, ap) upidosin, aq) SNAP-5089 (methyl 5-(N-(3-(4,4-diphenylpiperidin-1-yl)propyl)carbamoyl)-2,6-dimethyl-4(R)-(4-nitrophenyl)-1,4-dihydropyridine-3-carboxylate), ar) AIO-8507L, as) SL-890591 ((2-(3-(4-(5-chloro-2-methoxyphenyl)piperazin-1-yl)propylamino)pyrimidine-4-carboxamide fumarate), at) RS-100329 (5-methyl-3-(3-(4-(2-(2,2,2-trifluoroethoxy)phenyl)piperazin-1-yl)propyl) pyrimidine-2,4(1H,3H)-dione hydrochloride).

Details of the compounds may be found in the prior art.

Tamsulosin is preferably used in the form of the hydrochloride.

Preferably, au) finasteride and/or av) dutasteride are used as 5-alpha-reductase inhibitors.

The second component comprises one or more beta-3-adrenoreceptor agonists. This is (these are) preferably selected from the following group: 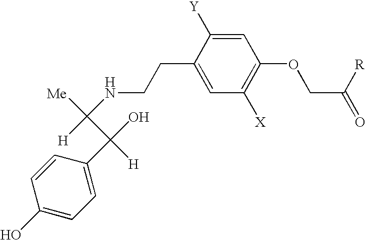
where
1) X=Br, Y=H, R=OH
2-[2-bromo-4-[2-[[(1S,2R)-2-hydroxy-2-(4-hydroxyphenyl)-1-methylethyl]amino]ethyl]phenoxy]acetic acid,
2) X=Cl, Y=H, R=OH
2-[2-chloro-4-[2-[[(1S,2R)-2-hydroxy-2-(4-hydroxyphenyl)-1-methylethyl]amino]ethyl]-phenoxy]acetic acid,
3) X=Y=Cl, R=OH
2-[2,5-dichloro-4-[2-[[(1S,2R)-2-hydroxy-2-(4-hydroxyphenyl)-1-methylethyl]amino]ethyl]phenoxy]acetic acid,
4) X=Y=H, R=OH
2-[4-[2-[[(1S,2R)-2-hydroxy-2-(4-hydroxyphenyl)-1-methylethyl]amino]ethyl]-2,5-dimethylphenoxy]acetic acid,
5) X=OH, Y=H, R=OH
2-[2-hydroxy-4-[2-[[(1S,2R)-2-hydroxy-2-(4-hydroxyphenyl)-1-methylethyl]amino]ethyl]phenoxy]acetic acid,
6) X=Cl, Y=H, R=OEt
ethyl-2-[2-chloro-4-[2-[[(1S,2R)-2-hydroxy-2-(4-hydroxyphenyl)-1-methylethyl]amino]ethyl]phenoxy]acetate,
7) X=Cl, Y=Cl, R=OEt
ethyl-2-[2,5-dichloro-4-[2-[[(1S,2R)-2-hydroxy-2-(4-hydroxyphenyl)-1-methylethyl]amino]ethyl]phenoxy]acetate,
8) X=Me; Y=Me, R=OEt (−)-ethyl-2-[4-(2-{[(1S,2R)-2-hydroxy-2-(4-hydroxyphenyl)-1-methylethyl]amino}ethyl)-2,5-dimethylphenyloxy]acetate,
9) X=Me, Y=Me, R=OH
(−)-2-[4-(2-{[(1S,2R)-2-hydroxy-2-(4-hydroxyphenyl)-1-methylethyl]amino}ethyl)-2,5-dimethylphenyloxy]acetic acid,

Details of the above-mentioned compounds 1 to 9 can be found in WO 00/02846.
10)

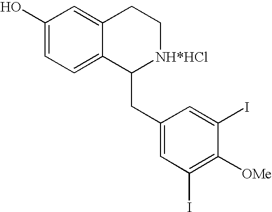

More information on this substance can be found in the J. Med. Chem. 44 (2001) 1456.
11)

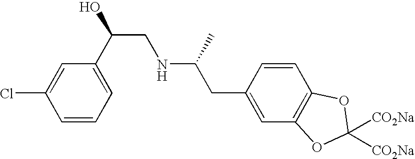

### Disodium-([R,R]-5-2-[[2-(3-chlorophenyl)-2-hydroxyethyl]-amino]propyl)-1,3-benzodioxole-2,2-dicarboxylate

More information on this substance can be found in J. Med. Chem. 44 (2001) 1456 or in the Journal of Urology 165 (2001) 240.
12)

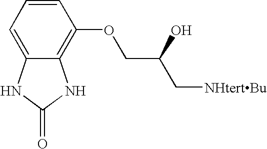

More information on this substance, which is also known as CGP 12177A, can be found in the Journal of Urology 165 (2001) 240 or in the J. Med. Chem. 44 (2001) 1456.
13)

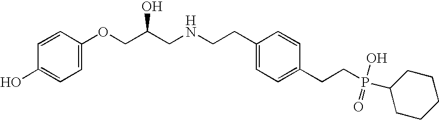

More information on this substance, which is also known as SB 226552, can be found in the J. Med. Chem. 44 (2001) 1456.
14)

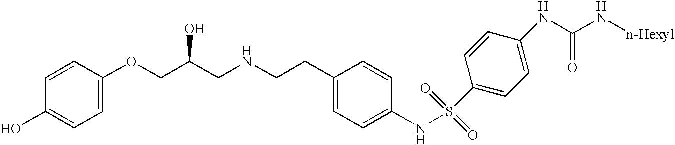

More information on this substance, which is also known as L755507, can be found in the J. Med. Chem. 44 (2001) 1456.
15)

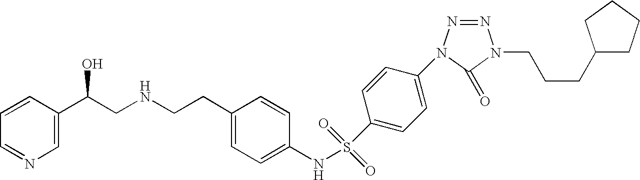

More information on this substance, which is also known as L 770664, can be found in the J. J. Med. Chem. 44 (2001) 1456.
16)

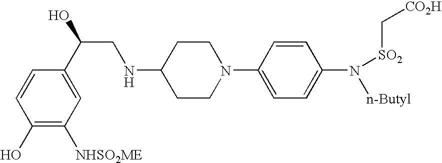

More information on this substance can be found in the J. Med. Chem. 44 (2001) 1456 or in the Bioorg. Med. Chem. Lett. 9 (2001) 2045.
17) 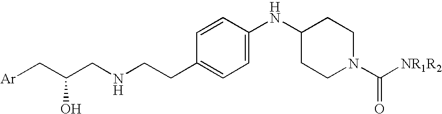
   where
Ar=4-OHPh-O—, R1=octyl, R2=H
Ar=4-OH,3-methylsulphonylamidophenyl-O, R1=2,5-diFbenzyl, R2=H
Ar=4-OH,3-methylsulphonylamidophenyl, R1=2,5-diFbenzyl, R2=H

More information on these substances can be found in the Bioorg. Med. Chem. Lett. 11 (2000) 3123.
18)

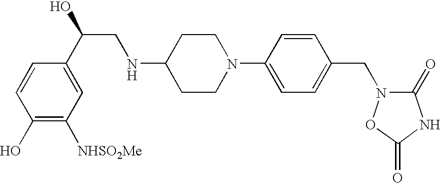

More information on this substance can be found in the Bioorg. Med. Chem. Lett. 11 (2001) 981.
19) 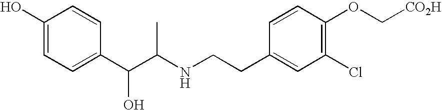
2-[2-chloro-4-(2-{[(1S,2R)-2-hydroxy-2-(4-hydroxyphenyl)-1-methylethyl]amino}ethyl)phenoxy]acetic acid

More information on this substance can be found in the Med. Chem. 46 (2003) 105.
20)

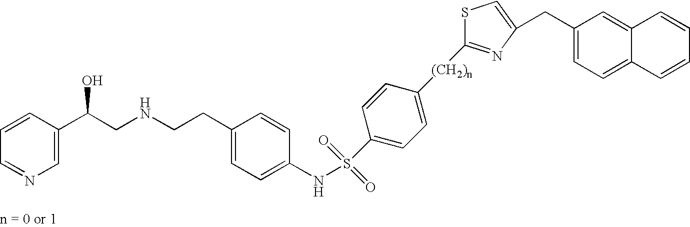

More information on this substance can be found in the Bioor. Med. Chem. Lett. 10 (2000) 1971.
21)

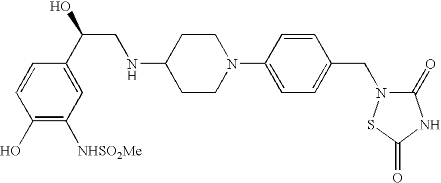

More information on this substance can be found in the Bioorg. Med. Chem. Lett. 11 (2001) 757.
22)

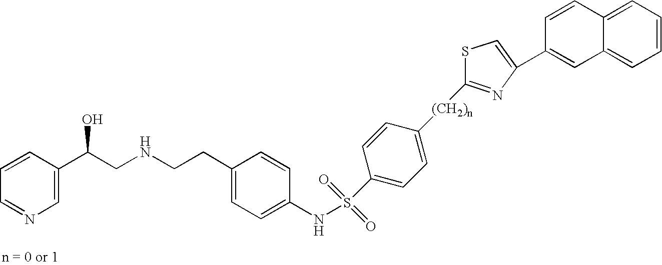

More information on this substance can be found in the Bioor. Med. Chem. Lett. 10 (2000) 1971.
23)

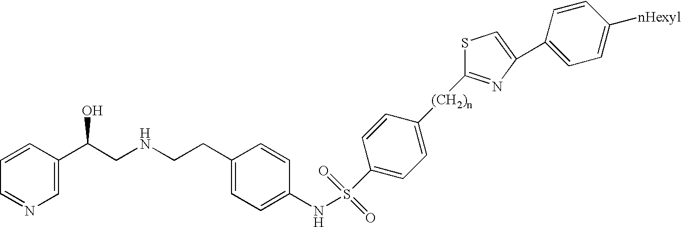

More information on this substance can be found in the Bioor. Med. Chem. Lett. 10 (2000) 1971.
24)

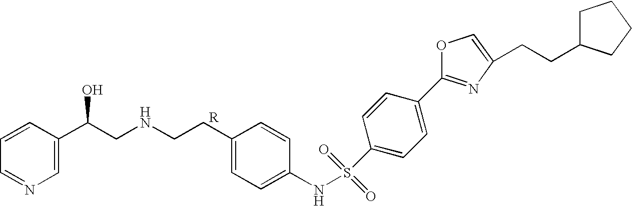

More information on this substance can be found in the Bioorg. Med. Chem. Lett. 10 (2000) 1531.
25) 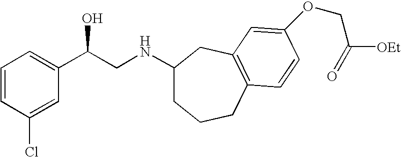
FK175
ethyl [R-(R*,S*)]-[[8-[[2-(3-chlorophenyl)-2-hydroxyethyl]amino]-6,7,8,9-tetrahydro-5H-benzocyclohepten-2-yl]oxy]-acetate, hydrochloride,
26) 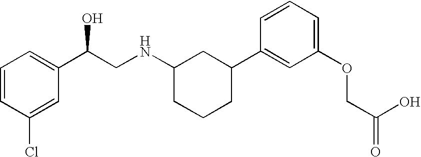
GS-332
[1S-[1α,3β(S*)]]-3-[3-[[2-(3-chlorophenyl)-2-hydroxyethyl]amino]cyclohexyl]phenoxy]-acetic acid, monosodium salt,
27)

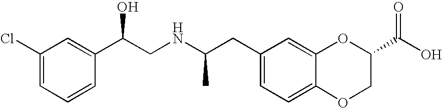

More information on this compound, also known as N-5984, can be found in the literature.
28) 2-(3-{[2-(3-chlorophenyl)-2R-hydroxyl-ethylamino]ethylamino}phenyl) furan-3-carboxylic acid. More information on this compound can be found in the literature.
29) 2-(3-{[2-(3-chlorophenyl)-2R-hydroxyl-ethylamino]ethylamino}phenyl) thiophene-3-carboxylic acid. Information on this compound can be found in the literature.
30)

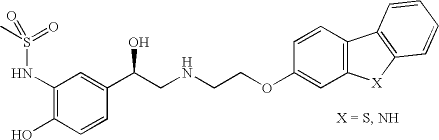

More information on this compound, also known as SB-418790, can be found in the literature.
31)

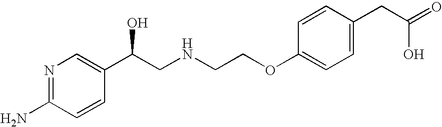

More information on this compound, also known as CP-331684, can be found in the literature.
32)

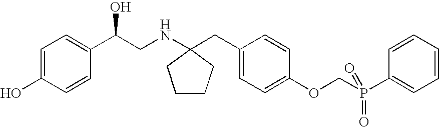

More information on this compound, also known as SB-251023, can be found in the literature.
33)

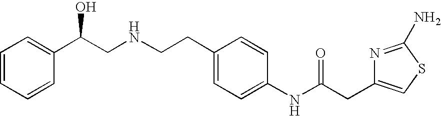

More information on this compound, (R)-2-(2-aminothiazol-4-yl)-4′-[2-[2-(hydroxy-2-phenylethyl)amino]ethyl]acetanilide, can be found in the literature WO 03/037881.
34)
(S)-4-[2-Hydroxy-3-[[2-[4-(5-carbamoyl-2-pyridyloxy)phenyl]-1,1-dimethylethyl]amino]-propoxy]-carbazole (LY 377604).
35)

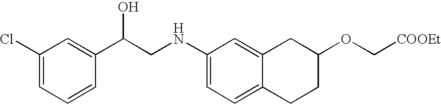

This compound is also known by the name SR 58611.

Most preferred are:
(−)-ethyl-2-[4-(2-{[(1S,2R)-2-hydroxy-2-(4-hydroxyphenyl)-1-methylethyl]amino}ethyl)-2,5-dimethylphenyloxy]acetate,
(−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride,
(−)-2-[4-(2-{[(1S,2R)-2-hydroxy-2-(4-hydroxyphenyl)-1-methylethyl]amino}ethyl)-2,5-dimethylphenyloxy]acetic acid,
   or other pharmacologically acceptable salts thereof.

Particularly interesting examples of beta-3-adrenoceptor agonists are: (−)-ethyl-2-[4-(2-{[(1S,2R)-2-hydroxy-2-(4-hydroxyphenyl)-1-methylethyl]amino}ethyl)-2,5-dimethylphenyloxy]acetate or (−)-2-[4-(2-{[(1S,2R)-2-hydroxy-2-(4-hydroxyphenyl)-1-methylethyl]-amino}ethyl)-2,5-dimethylphenyloxy]acetic acid, the enantiomers, other diastereoisomers thereof, and pharmacologically active salts thereof.

These compounds are disclosed in WO 00/02846 or WO 2003024916.

These last two compounds are represented by the following formula II, which should take precedence over the specified name, in the event of any inconsistencies: 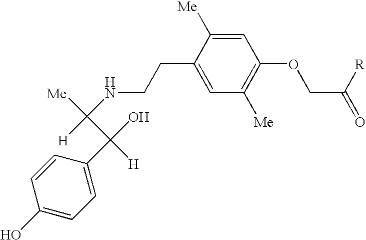
where R=O-ethyl: (−)-ethyl-2-[4-(2-{[(1S,2R)-2-hydroxy-2-(4-hydroxyphenyl)-1-methylethyl]amino}ethyl)-2,5-dimethylphenyloxy]acetate, preferably the monohydrate,
where R=OH: (−)-2-[4-(2-{[(1S,2R)-2-hydroxy-2-(4-hydroxyphenyl)-1-methylethyl]-amino}ethyl)-2,5-dimethylphenyloxy]acetic acid.

Particularly preferred combinations comprise a combination of: (a) tamsulosin, either in its enantiomeric or racemic form, or pharmacologically acceptable salts thereof, or any active metabolites thereof, and (b) at least one of the following compounds: (−)-ethyl-2-[4-(2-{[(1S,2R)-2-hydroxy-2-(4-hydroxyphenyl)-1-methylethyl]amino}ethyl)-2,5-dimethylphenyloxy]acetate, (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride, (−)-2-[4-(2-{[(1S,2R)-2-hydroxy-2-(4-hydroxyphenyl)-1-methylethyl]amino}ethyl)-2,5-dimethylphenyloxy]acetic acid, or any other pharmacologically acceptable salts thereof, or any active metabolites thereof.

Other particularly preferred combinations comprise a combination of: (a) finasteride, either in its enantiomeric or racemic form, or pharmacologically acceptable salts thereof, or any active metabolites thereof, and (b) at least one of the following compounds: (−)-ethyl-2-[4-(2-{[(1S,2R)-2-hydroxy-2-(4-hydroxyphenyl)-1-methylethyl]amino}ethyl)-2,5-dimethylphenyloxy]acetate, (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride, (−)-2-[4-(2-{[(1S,2R)-2-hydroxy-2-(4-hydroxyphenyl)-1-methylethyl]amino}ethyl)-2,5-dimethylphenyloxy]acetic acid, or any other pharmacologically acceptable salts thereof, or any active metabolites thereof.

Other particularly preferred combinations comprise a combination of: (a) dutasteride, either in its enantiomeric or racemic form, or pharmacologically acceptable salts thereof, or any active metabolites thereof, and (b) at least one of the following compounds: (−)-ethyl-2-[4-(2-{[(1S,2R)-2-hydroxy-2-(4-hydroxyphenyl)-1-methylethyl]amino}ethyl)-2,5-dimethylphenyloxy]acetate, (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride, (−)-2-[4-(2-{[(1S,2R)-2-hydroxy-2-(4-hydroxyphenyl)-1-methylethyl]amino}ethyl)-2,5-dimethylphenyloxy]acetic acid, or any other pharmacologically acceptable salts thereof, or any active metabolites thereof.

Other particularly preferred triple combinations comprise a combination of: (a) tamsulosin, or the hydrochloride thereof and finasteride, either in its enantiomeric or racemic form, or pharmacologically acceptable salts thereof, or any active metabolites thereof, and (b) at least one of the following compounds: (−)-ethyl-2-[4-(2-{[(1S,2R)-2-hydroxy-2-(4-hydroxyphenyl)-1-methylethyl]amino}ethyl)-2,5-dimethylphenyloxy]acetate, (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride, (−)-2-[4-(2-{[(1S,2R)-2-hydroxy-2-(4-hydroxyphenyl)-1-methylethyl]amino}ethyl)-2,5-dimethylphenyloxy]acetic acid, or any other pharmacologically acceptable salts thereof, or any active metabolites thereof.

It is expressly pointed out that the invention includes every one of the following combinations: (aa, 1); (ab, 1); (ac, 1); (ad, 1); (ae, 1); (af, 1); (ag, 1); (ah, 1); (ai, 1); (aj, 1); (ak, 1); (al, 1); (am, 1); (an, 1); (ao, 1); (ap, 1); (aq, 1); (ar, 1); (as, 1); (at, 1); (au, 1); (av, 1); (aa, 2); (ab, 2); (ac, 2); (ad, 2); (ae, 2); (af, 2); (ag, 2); (ah, 2); (ai, 2); (aj, 2); (ak, 2); (al, 2); (am, 2); (an, 2); (ao, 2); (ap, 2); (aq, 2); (ar, 2); (as, 2); (at, 2); (au, 2); (av, 2); (aa, 3); (ab, 3); (ac, 3); (ad, 3); (ae, 3); (af, 3); (ag, 3); (ah, 3); (ai, 3); (aj, 3); (ak, 3); (al, 3); (am, 3); (an, 3); (ao, 3); (ap, 3); (aq, 3); (ar, 3); (as, 3); (at, 3); (au, 3); (av, 3); (aa, 4); (ab, 4); (ac, 4); (ad, 4); (ae, 4); (af, 4); (ag, 4); (ah, 4); (ai, 4); (aj, 4); (ak, 4); (al, 4); (am, 4); (an, 4); (ao, 4); (ap, 4); (aq, 4); (ar, 4); (as, 4); (at, 4); (au, 4); (av, 4); (aa, 5); (ab, 5); (ac, 5); (ad, 5); (ae, 5); (af, 5); (ag, 5); (ah, 5); (ai, 5); (aj, 5); (ak, 5); (al, 5); (am, 5); (an, 5); (ao, 5); (ap, 5); (aq, 5); (ar, 5); (as, 5); (at, 5); (au, 5); (av, 5); (aa, 6); (ab, 6); (ac, 6); (ad, 6); (ae, 6); (af, 6); (ag, 6); (ah, 6); (ai, 6); (aj, 6); (ak, 6); (al, 6); (am, 6); (an, 6); (ao, 6); (ap, 6); (aq, 6); (ar, 6); (as, 6); (at, 6); (au, 6); (av, 6); (aa, 7); (ab, 7); (ac, 7); (ad, 7); (ae, 7); (af, 7); (ag, 7); (ah, 7); (ai, 7); (aj, 7); (ak, 7); (al, 7); (am, 7); (an, 7); (ao, 7); (ap, 7); (aq, 7); (ar, 7); (as, 7); (at, 7); (au, 7); (av, 7); (aa, 8); (ab, 8); (ac, 8); (ad, 8); (ae, 8); (af, 8); (ag, 8); (ah, 8); (ai, 8); (aj, 8); (ak, 8); (al, 8); (am, 8); (an, 8); (ao, 8); (ap, 8); (aq, 8); (ar, 8); (as, 8); (at, 8); (au, 8); (av, 8); (aa, 9); (ab, 9); (ac, 9); (ad, 9); (ae, 9); (af, 9); (ag, 9); (ah, 9); (ai, 9); (aj, 9); (ak, 9); (al, 9); (am, 9); (an, 9); (ao, 9); (ap, 9); (aq, 9); (ar, 9); (as, 9); (at, 9); (au, 9); (av, 9); (aa, 10); (ab, 10); (ac, 10); (ad, 10); (ae, 10); (af, 10); (ag, 10); (ah, 10); (ai, 10); (aj, 10); (ak, 10); (al, 10); (am, 10); (an, 10); (ao, 10); (ap, 10); (aq, 10); (ar, 10); (as, 10); (at, 10); (au, 10); (av, 10); (aa, 11); (ab, 11); (ac, 11); (ad, 11); (ae, 11); (af, 11); (ag, 11); (ah, 11); (ai, 11); (aj, 11); (ak, 11); (al, 11); (am, 11); (an, 11); (ao, 11); (ap, 11); (aq, 11); (ar, 11); (as, 11); (at, 11); (au, 11); (av, 11); (aa, 12); (ab, 12); (ac, 12); (ad, 12); (ae, 12); (af, 12); (ag, 12); (ah, 12); (ai, 12); (aj, 12); (ak, 12); (al, 12); (am, 12); (an, 12); (ao, 12); (ap, 12); (aq, 12); (ar, 12); (as, 12); (at, 12); (au, 12); (av, 12); (aa, 13); (ab, 13); (ac, 13); (ad, 13); (ae, 13); (af, 13); (ag, 13); (ah, 13); (ai, 13); (aj, 13); (ak, 13); (al, 13); (am, 13); (an, 13); (ao, 13); (ap, 13); (aq, 13); (ar, 13); (as, 13); (at, 13); (au, 13); (av, 13); (aa, 14); (ab, 14); (ac, 14); (ad, 14); (ae, 14); (af, 14); (ag, 14); (ah, 14); (ai, 14); (aj, 14); (ak, 14); (al, 14); (am, 14); (an, 14); (ao, 14); (ap, 14); (aq, 14); (ar, 14); (as, 14); (at, 14); (au, 14); (av, 14); (aa, 15); (ab, 15); (ac, 15); (ad, 15); (ae, 15); (af, 15); (ag, 15); (ah, 15); (ai, 15); (aj, 15); (ak, 15); (al, 15); (am, 15); (an, 15); (ao, 15); (ap, 15); (aq, 15); (ar, 15); (as, 15); (at, 15); (au, 15); (av, 15); (aa, 16); (ab, 16); (ac, 16); (ad, 16); (ae, 16); (af, 16); (ag, 16); (ah, 16); (ai, 16); (aj, 16); (ak, 16); (al, 16); (am, 16); (an, 16); (ao, 16); (ap, 16); (aq, 16); (ar, 16); (as, 16); (at, 16); (au, 16); (av, 16); (aa, 17); (ab, 17); (ac, 17); (ad, 17); (ae, 17); (af, 17); (ag, 17); (ah, 17); (ai, 17); (aj, 17); (ak, 17); (al, 17); (am, 17); (an, 17); (ao, 17); (ap, 17); (aq, 17); (ar, 17); (as, 17); (at, 17); (au, 17); (av, 17); (aa, 18); (ab, 18); (ac, 18); (ad, 18); (ae, 18); (af, 18); (ag, 18); (ah, 18); (ai, 18); (aj, 18); (ak, 18); (al, 18); (am, 18); (an, 18); (ao, 18); (ap, 18); (aq, 18); (ar, 18); (as, 18); (at, 18); (au, 18); (av, 18); (aa, 19); (ab, 19); (ac, 19); (ad, 19); (ae, 19); (af, 19); (ag, 19); (ah, 19); (ai, 19); (aj, 19); (ak, 19); (al, 19); (am, 19); (an, 19); (ao, 19); (ap, 19); (aq, 19); (ar, 19); (as, 19); (at, 19); (au, 19); (av, 19); (aa, 20); (ab, 20); (ac, 20); (ad, 20); (ae, 20); (af, 20); (ag, 20); (ah, 20); (ai, 20); (aj, 20); (ak, 20); (al, 20); (am, 20); (an, 20); (ao, 20); (ap, 20); (aq, 20); (ar, 20); (as, 20); (at, 20); (au, 20); (av, 20); (aa, 21); (ab, 21); (ac, 21); (ad, 21); (ae, 21); (af, 21); (ag, 21); (ah, 21); (ai, 21); (aj, 21); (ak, 21); (al, 21); (am, 21); (an, 21); (ao, 21); (ap, 21); (aq, 21); (ar, 21); (as, 21); (at, 21); (au, 21); (av, 21); (aa, 22); (ab, 22); (ac, 22); (ad, 22); (ae, 22); (af, 22); (ag, 22); (ah, 22); (ai, 22); (aj, 22); (ak, 22); (al, 22); (am, 22); (an, 22); (ao, 22); (ap, 22); (aq, 22); (ar, 22); (as, 22); (at, 22); (au, 22); (av, 22); (aa, 23); (ab, 23); (ac, 23); (ad, 23); (ae, 23); (af, 23); (ag, 23); (ah, 23); (ai, 23); (aj, 23); (ak, 23); (al, 23); (am, 23); (an, 23); (ao, 23); (ap, 23); (aq, 23); (ar, 23); (as, 23); (at, 23); (au, 23); (av, 23); (aa, 24); (ab, 24); (ac, 24); (ad, 24); (ae, 24); (af, 24); (ag, 24); (ah, 24); (ai, 24); (aj, 24); (ak, 24); (al, 24); (am, 24); (an, 24); (ao, 24); (ap, 24); (aq, 24); (ar, 24); (as, 24); (at, 24); (au, 24); (av, 24); (aa, 25); (ab, 25); (ac, 25); (ad, 25); (ae, 25); (af, 25); (ag, 25); (ah, 25); (ai, 25); (aj, 25); (ak, 25); (al, 25); (am, 25); (an, 25); (ao, 25); (ap, 25); (aq, 25); (ar, 25); (as, 25); (at, 25); (au, 25); (av, 25); (aa, 26); (ab, 26); (ac, 26); (ad, 26); (ae, 26); (af, 26); (ag, 26); (ah, 26); (ai, 26); (aj, 26); (ak, 26); (al, 26); (am, 26); (an, 26); (ao, 26); (ap, 26); (aq, 26); (ar, 26); (as, 26); (at, 26); (au, 26); (av, 26); (aa, 27); (ab, 27); (ac, 27); (ad, 27); (ae, 27); (af, 27); (ag, 27); (ah, 27); (ai, 27); (aj, 27); (ak, 27); (al, 27); (am, 27); (an, 27); (ao, 27); (ap, 27); (aq, 27); (ar, 27); (as, 27); (at, 27); (au, 27); (av, 27); (aa, 28); (ab, 28); (ac, 28); (ad, 28); (ae, 28); (af, 28); (ag, 28); (ah, 28); (ai, 28); (aj, 28); (ak, 28); (al, 28); (am, 28); (an, 28); (ao, 28); (ap, 28); (aq, 28); (ar, 28); (as, 28); (at, 28); (au, 28); (av, 28); (aa, 29); (ab, 29); (ac, 29); (ad, 29); (ae, 29); (af, 29); (ag, 29); (ah, 29); (ai, 29); (aj, 29); (ak, 29); (al, 29); (am, 29); (an, 29); (ao, 29); (ap, 29); (aq, 29); (ar, 29); (as, 29); (at, 29); (au, 29); (av, 29); (aa, 30); (ab, 30); (ac, 30); (ad, 30); (ae, 30); (af, 30); (ag, 30); (ah, 30); (ai, 30); (aj, 30); (ak, 30); (al, 30); (am, 30); (an, 30); (ao, 30); (ap, 30); (aq, 30); (ar, 30); (as, 30); (at, 30); (au, 30); (av, 30); (aa, 31); (ab, 31); (ac, 31); (ad, 31); (ae, 31); (af, 31); (ag, 31); (ah, 31); (ai, 31); (aj, 31); (ak, 31); (al, 31); (am, 31); (an, 31); (ao, 31); (ap, 31); (aq, 31); (ar, 31); (as, 31); (at, 31); (au, 31); (av, 31); (aa, 32); (ab, 32); (ac, 32); (ad, 32); (ae, 32); (af, 32); (ag, 32); (ah, 32); (ai, 32); (aj, 32); (ak, 32); (al, 32); (am, 32); (an, 32); (ao, 32); (ap, 32); (aq, 32); (ar, 32); (as, 32); (at, 32); (au, 32); (av, 32); (aa, 33); (ab, 33); (ac, 33); (ad, 33); (ae, 33); (af, 33); (ag, 33); (ah, 33); (ai, 33); (aj, 33); (ak, 33); (al, 33); (am, 33); (an, 33); (ao, 33); (ap, 33); (aq, 33); (ar, 33); (as, 33); (at, 33); (au, 33); (av, 33); (aa, 34); (ab, 34); (ac, 34); (ad, 34); (ae, 34); (af, 34); (ag, 34); (ah, 34); (ai, 34); (aj, 34); (ak, 34); (al, 34); (am, 34); (an, 34); (ao, 34); (ap, 34); (aq, 34); (ar, 34); (as, 34); (at, 34); (au, 34); (av, 34); (aa, 35); (ab, 35); (ac, 35); (ad, 35); (ae, 35); (af, 35); (ag, 35); (ah, 35); (ai, 35); (aj, 35); (ak, 35); (al, 35); (am, 35); (an, 35); (ao, 35); (ap, 35); (aq, 35); (ar, 35); (as, 35); (at, 35); (au, 35); (av, 35).

b) Dosage

In order to determine the optimum dose of the two active substances for urinary incontinence, various basic conditions have to be taken into consideration such as for example the age and body weight of the patient, the nature and stage of the disease and the potency of the compound. This is deemed to be within the capabilities of the skilled man, and the existing literature on the components can be consulted in order to arrive at the optimum dose. The doses specified relate to the dosage after the end of the adjustment phase.

The doses given hereinafter expressly include all the numerical values, both whole numbers and fractions, within the range specified. The data relate to adults. Pediatric doses may be lower.

Doses administered more than once a day or twice a day (e.g. 3, 4, 5, or 6 times a day) are also expressly included herein.

The choice of dosage of this first component (1) is that which gives relief to the patient. In some cases a smaller amount than specified may be sufficient while in other cases a larger total amount may be required.

The total daily dose may be taken in one go or in several portions depending on the treatment plan. The treatment plan may also prescribe intervals of longer than one day between the doses.

The preferred dose of the alpha antagonist for humans is between 0.001 mg and 5 g per day, preferably between 0.001 mg and 100 mg and most preferably between 0.1 mg and 50 mg.

Desirably, the daily dose of the combination according to the invention in the case of the active substance tamsulosin, as component (a) contains the latter in an amount of from about 0.05 mg to about 5 mg. Preferably, each dose of the component contains about 0.1 to about 1 mg of the active substance. This dosage form enables the total daily dose to be taken in half or whole doses, all at once or in several portions. Doses taken more than once a day or twice a day (e.g. 3, 4, 5 or 6 times a day) are also expressly included.

The average adult daily dose of the other possible examples of alpha antagonist is as follows.

The average daily dose of the component (mg/day/patient) is:
alfuzosin (1 mg to 15 mg, preferably approx. 7.5 mg), bunazosin (0.5 mg to 20 mg preferably 5.5 mg), doxazosin 0.5 to 15 mg, preferably up to 4 mg), indoramin (1 to 50 mg, preferably to 25 mg), naftopidil (1 mg to 100 mg, preferably less than 50 mg), prazosin (1 mg to 10 mg), terazosin (0,1 mg to 5 mg, preferably 2 mg), urapidil (10 mg to 150 mg, preferably 30 mg to 90 mg).

If a 5-alpha-reductase inhibitor is used, the average daily dose for an adult is 0.1 mg to 10 mg, preferably 5 mg of finasteride, and 0.01 mg to 2 mg, preferably 0.5 mg of dutasteride.

The doses and the treatment plan (i.e. one, two, three, or more doses per day) of the second component depend on the factors to which reference was made in conjunction with the choice of dosage for the first component.

The average daily dose for adults of the second component (beta-3-agonist) is about 1 mg to 1000 mg, preferably 10 mg to about 750 mg per day, preferably 50 to 500 mg, more preferably 80 to 200 mg, administered in one or more doses.

c) Formulations

The compositions of the present invention may conveniently be administered in a pharmaceutical composition which contains the active components in combination with a suitable carrier. Such pharmaceutical compositions may be prepared by methods and contain carriers which are well known in the art. Generally recognised textbooks are available to the skilled man for this purpose.

The compositions of the present invention may be administered parenterally (e.g. by intravenous, intraperitoneal, subcutaneous or intramuscular injection), topically, orally, intranasally, transdermally, rectally, by pulmonary or nasal inhalation, oral administration being particularly preferred. Of the oral formulations, those which are resistant to gastric juices are preferred. Therefore, capsules or tablets resistant to gastric juices are preferred, and in both cases this may be achieved with a coating which is resistant to gastric juices. The skilled man will find instructions for formulations resistant to gastric juices in the prior art.

Various formulating options are described below. The skilled man may choose a suitable formulation from them.

For oral therapeutic administration the composition according to the invention may be combined with one or more carriers and used in the form of tablets for swallowing, buccal tablets, sublingual tablets, sugar-coated tablets, sprays, powders, pastilles, coated tablets, granules, capsules, elixirs, suspensions, solutions, syrups, lozenges, chewing gums, foods, and the like.

A powder may be prepared for example by grinding the particles of active substance to a suitable size.

Dilute powders may be prepared by finely grinding the powdered substance with a non-toxic carrier material such as lactose and delivering it as a powder. Other suitable carrier materials for this purpose are other carbohydrates, such as starch or mannitol. These powders may optionally contain flavorings, preservatives, dispersing agents, colorings and other pharmacological adjuvants.

Capsules may be prepared from a powder of the kind described above or other powders, which are placed in a capsule, preferably a gelatin capsule, and the capsule is then sealed.

It is also possible for lubricants known from the prior art to be introduced into the capsule or used to seal the two parts of the capsule. The efficacy of a capsule when taken orally can be increased by the addition of disintegrating or solubilizing substances such as, for example, carboxymethylcelluose, carboxymethylcellulose calcium, low-substituted hydroxypropylcellulose, calcium carbonate, sodium carbonate, and other substances. The active substance may be present in the capsule not only as a solid but also in suspended form, for example, in vegetable oil, polyethyleneglycol, or glycerol using surface-active substances, etc.

Tablets may be prepared by compressing the powdered mixture and then processing it into granules, for example. The tablets may contain various excipients such as e.g. starches, lactose, sucrose, glucose, sodium chloride, urea for tablets for dissolving or injecting, amylose, various types of cellulose as described above, and others. Glycerol or starch may be used as a moisture retaining agent.

The disintegrants used may be, for example, starch, alginic acid, calcium alginate, pectic acid, powdered agar-agar, formaldehyde gelatine, calcium carbonate, sodium bicarbonate, magnesium peroxide, and amylose.

Anti-disintegrants or solution retardants which may be used include, for example, sucrose, stearin, solid paraffin (preferably with a melting point in the range from 50-52° C.), cocoa butter, and hydrogenated fats.

Other disintegrants may be: corn starch, potato starch, alginic acid, and the like.

Suitable absorption accelerators include, inter alia, quaternary ammonium compounds, sodium lauryl sulphate, and saponins.

Ether may be used, for example, as a binder distributor, and cetyl alcohol, glycerol monostearate, starch, corn starch, lactose, wetting agents (e.g. aerosol OT, Pluronics, Tweens), gum tragacanth, gum arabic, gelatin, and others may be used as hydrophilising agents or disintegration accelerators.

Sucrose, fructose, lactose, or aspartame may be used as sweeteners, while peppermint, wintergreen oil, cherry flavoring, etc., may be used as flavoring agents.

The following may also be generally used as additional excipients: fumed silica (e.g., Aerosil®), sodium dioctyl sulfosuccinate (e.g., Aerosol® OT), ethylcellulose, ion-exchange resin (e.g., Amberlite® XE-88), corn starch, Amisterol, amylose, microcrystalline-cellulose (e.g., Avicel®), bentonite, calcium sulphate, polyethylene glycol (e.g., Carbowax® 4000 and 6000), carrageen, castor wax, cellulose, microcrystalline cellulose, crospovidone, dextrane, dextrin, dicalcium phosphate, pharmaceutical tablet base, kaolin, lactose (USP), lactosil, magnesium stearate, mannitol, granular mannitol N.F. methylcellulose, fatty acid esters (e.g., Miglyol® 812 neutral oil), powdered milk, powdered sugar, nal-tab, crystalline sorbitol, povidone (e.g., Plasdone®), polyethyleneglycols, polyvinylacetate phthalate, polyvinylpyrrolidone, atomized glyceryl palmitostearate (e.g., Precirol®), neat's foot oil (hydrogenated), melting tablet base, silicone, stabiline, pregelatinized starch (e.g., Starx® 1500), silica (e.g., Syloid®), Waldhof tablet base, tablettol, talcum cetylatum and stearatum, metal soaps, fructose, and cellulose ethers (e.g., Tylose®). The tabletting excipient K (M25) is particularly suitable, and also complies with the requirements of the following pharmacopoeias: DAB, Ph. Eur., BP, and NF.

Other excipients which may be used can be found in the Examples, but other excipients known from the prior art may also be used.

The tablets may be produced by direct compression, for example.

It is also possible to prepare other formulations for oral administration such as solutions, syrups, elixirs etc. If desired the compound may be micro-encapsulated.

Parenteral administration may be achieved by dissolving the compound in a liquid and injecting it by subcutaneous, intramuscular, or intravenous route. Suitable solvents include, for example, water or oily media.

In order to prepare suppositories, the compound may be formulated with low-melting and water-soluble or water-insoluble materials, such as polyethylene glycol, cocoa butter, higher esters (for example moerysthyl, palmitate), or mixtures thereof.

The above list is provided solely by way of example and a skilled man might consider other excipients.

Various other materials may be provided as coatings or for modifying the physical form of the solid dosage units in some other way. For example, tablets, pills, or capsules may be coated with gelatin, wax, shellac, or sugar, and the like. As already mentioned, formulations resistant to gastric juices are preferred for the oral preparations. Therefore, gastric juice-resistant coatings are preferred for tablets or capsules. In the case of a syrup or elixir, sucrose or fructose may be used as the sweetener, methyl- and propylparaben may be present as preservatives, and a coloring and a flavoring agent, such as cherry or orange flavor, may also be present.

The excipients mentioned above are not restricted to the use of the formulation in connection with which they have been mentioned but may also be applied to the other formulations.

Naturally, any material used in the preparations of any of these dosage units must be pharmaceutically acceptable and substantially non-toxic in the amounts used. In addition, the active components may be incorporated in preparations with delayed release and devices which, without being restricted thereto, include those based on osmotic pressures, in order to achieve the desired release profile. One-a-day formulations for each of the active components are particularly included.

Compositions and preparations of this kind should contain at least 0.001% of active compound. The percentage of the compositions and preparations may naturally vary and may appropriately make up between 0.1 and about 100% of the weight of a given dosage unit. The quantity of active compound in therapeutically useful compositions of this kind is such that an effective dose is present.

The composition according to the invention which contains the two active components may be administered in the same physical form or at the same time in accordance with the dosages described above and in the administration carriers described above. The dosages for each active component may be measured separately and may be administered as a single combined dose or separately. They may be given at the same time or at different times provided that both active ingredients come to act in the patient at some time over a 24 hour period. It is preferable if the two components act in such a way as to achieve an effect which is better than the individual activity in each case. Simultaneous or coincident administration means that the patient takes one drug within about five minutes of taking the other drug. For ease of handling it is preferable to use formulations in which the two drugs are given to the patient close together and typically at the same time.

d) Indications

The pharmaceutical composition may preferably be used to treat or prevent, inter alia, each of the syndromes mentioned below, as an individual syndrome and in conjunction with another of the syndromes mentioned, without being restricted thereto: prostate diseases, such as benign prostatic hyperplasia, prostatitis, particularly chronic abacterial prostatitis, of neurogenic, muscular, or bacterial origin, chronic pain syndrome of the pelvis, pelvic myoneuropathy, prostatodynia or prostatopathy, functional bladder disorders, such as urinary incontinence, particularly stress incontinence, urge incontinence, mixed incontinence, or overactive bladder, of neurogenic or non-neurogenic origin and further sub-indications thereof.

Preferably, the invention is used when one of the above-mentioned prostate diseases and one of the above-mentioned functional bladder disorders are both present at the same time.

Thus, the invention includes both those syndromes whose cause is dysfunction or disease of an organ and those which can be attributed to diseases or disorders of the central nervous system. The composition according to the invention may lead to alleviation of the symptoms of the disease(s) and/or the underlying cause of the disease is treated.

Thus, a further embodiment of the present invention comprises using the composition according to the invention to prepare a drug for treating or preventing any of the indications mentioned in the preceding paragraph.

The above diseases or disorders are treated by administering a therapeutically effective amount of the composition according to the invention to a mammal. In most cases this is a human being but the treatment of farm animals (e.g. cattle) and domestic animals (e.g. dogs, cats and horses) is also expressly covered. For use in veterinary medicine, the dosages used may be different from those specified herein.

It is expected that the new composition will provide rapid relief for those suffering from the above diseases and disorders with a minimum amount of harmful side effects.

### e) EXAMPLES

The invention is illustrated by the following non-restrictive Examples.

Particularly preferred combinations are
Tamsulosin hydrochloride and (−)-ethyl-2-[4-(2-{[(1S,2R)-2-hydroxy-2-(4-hydroxyphenyl)-1-methylethyl]amino}ethyl)-2,5-dimethylphenyloxy]acetate.
Tamsulosin hydrochloride and (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride.
Tamsulosin hydrochloride and (−)-2-[4-(2-{[(1S,2R)-2-hydroxy-2-(4-hydroxyphenyl)-1-methylethyl]amino}ethyl)-2,5-dimethylphenyloxy]acetic acid.
Tamsulosin hydrochloride and (−)-2-[4-(2-{[(1S,2R)-2-hydroxy-2-(4-hydroxyphenyl)-1-methylethyl]amino}ethyl)-2,5-dimethylphenyloxy]acetic acid-monohydrochloride.
Finasteride and (−)-ethyl-2-[4-(2-{[(1S,2R)-2-hydroxy-2-(4-hydroxyphenyl)-1-methylethyl]amino}ethyl)-2,5-dimethylphenyloxy]acetate.
Finasteride and (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride.
Finasteride and (−)-2-[4-(2-{[(1S,2R)-2-hydroxy-2-(4-hydroxyphenyl)-1-methylethyl]-amino}ethyl)-2,5-dimethylphenyloxy]acetic acid.
Finasteride and (−)-2-[4-(2-{[(1S,2R)-2-hydroxy-2-(4-hydroxyphenyl)-1-methylethyl]-amino}ethyl)-2,5-dimethylphenyloxy]acetic acid-monohydrochloride.
Dutasteride and (−)-ethyl-2-[4-(2-{[(1S,2R)-2-hydroxy-2-(4-hydroxyphenyl)-1-methylethyl]amino}ethyl)-2,5-dimethylphenyloxy]acetate.
Dutasteride and (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride.
Dutasteride and (−)-2-[4-(2-{[(1S,2R)-2-hydroxy-2-(4-hydroxyphenyl)-1-methylethyl]-amino}ethyl)-2,5-dimethylphenyloxy]acetic acid.
Dutasteride and (−)-2-[4-(2-{[(1S,2R)-2-hydroxy-2-(4-hydroxyphenyl)-1-methylethyl]-amino}ethyl)-2,5-dimethylphenyloxy]acetic acid-monohydrochloride.

Now that the invention has been described in detail and with reference to the preferred embodiments thereof, it is obvious that modifications and changes are possible without departing from the scope of the appended claims.

### Example 1

### Composition comprising (−)-ethyl-2-[4-(2-{[(1S,2R)-2-hydroxy-2-(4-hydroxyphenyl)-1-methylethyl]amino}ethyl)-2,5-dimethylphenyloxy]acetate/tamsulosin: Delayed-Release Capsule 80 mg/0.367 mg.

Pellets

| Ingredients | mg/capsule |
|---|---|
| (−)-ethyl-2-[4-(2-{[(1S,2R)-2-hydroxy-2- | 87.280 |
| (4-hydroxyphenyl)-1-methyl-ethyl]amino}ethyl)- | |
| 2,5-dimethylphenyloxy]acetate- | |
| monohydrochloride | |
| Tamsulosin hydrochloride | 0.400 |
| microcrystalline cellulose | 322.600 |
| poly[(methacrylic acid)(ethyl acrylate)] (1:1) | 56.000 |
| purified water | (q.s.) |

Gastric Juice-Resistant Coating

| Ingredients | mg/capsule |
|---|---|
| poly[(methacrylic acid)(ethyl acrylate)] (1:1) | 8.000 |
| triacetin | 1.320 |
| purified water | (q.s.) |

Final Mixture

| Ingredients | mg/capsule |
|---|---|
| coated pellets | 475.600 |
| talc | 1.200 |
| calcium stearate | 1.200 |

Capsule

| Ingredients | mg/capsule |
|---|---|
| Final mixture | 478.000 |
| hard gelatine capsule (size 1) | 82.000 |
| Total weight of the delayed-release capsule | 560.000 |

1. Pharmaceutical composition comprising: (a) a first active agent comprising a pharmaceutically effective amount of one or more alpha antagonists or 5-alpha reductase inhibitors, or a pharmacologically acceptable salt, enantiomer, diastereomer, tautomer, or metabolite thereof, and (b) a second active agent comprising a pharmaceutically effective amount of one or more beta-3-adrenoceptor agonists or a pharmacologically acceptable salt, enantiomer, diastereomer, tautomer, or metabolite thereof.

2. Pharmaceutical composition according to claim 1, wherein the first active agent is selected from the group consisting of: tamsulosin, tamsulosin hydrochloride, alfuzosin, bunazosin, doxazosin, indoramin, naftopidil, prazosin, terazosin, urapidil, silodosin, moxisylyte, metazosin, fiduxosin, upidosin, methyl 5-(N-(3-(4,4-diphenylpiperidin-1-yl)propyl)carbamoyl)-2,6-dimethyl-4(R)-(4-nitrophenyl)-1,4-dihydropyridin-3-carboxylate, AIO-8507L, 2-(3-(4-(5-chloro-2-methoxyphenyl)piperazin-1-yl)propylamino)pyrimidin-4-carboxamide fumarate), 5-methyl-3-(3-(4-(2-(2,2,2-trifluoroethoxy)phenyl)piperazin-1-yl)propyl)pyrimidin-2,4(1H,3H)-dione hydrochloride, and pharmacologically acceptable salts, enantiomers, diastereomers, tautomers, and metabolites thereof, and mixtures thereof.

3. Pharmaceutical composition according to claim 1, wherein the first active agent is selected from the group consisting of: tamsulosin, tamsulosin hydrochloride, finasteride, dutasteride, and pharmacologically acceptable salts, enantiomers, diastereomers, tautomers, and metabolites thereof, and mixtures thereof.

4. Pharmaceutical composition according to one of claim 1, wherein the second active agent is selected from the group consisting of:
(−)-ethyl-2-[4-(2-{[(1S,2R)-2-hydroxy-2-(4-hydroxyphenyl)-1-methylethyl]-amino}-ethyl)-2,5-dimethylphenyloxy]acetate,
(−)-ethyl-2-[4-(2-{[(1S,2R)-2-hydroxy-2-(4-hydroxyphenyl)-1-methylethyl]-amino}-ethyl)-2,5-dimethylphenyloxy]acetate-monohydrochloride, and
(−)-2-[4-(2-{[(1S,2R)-2-hydroxy-2-(4-hydroxyphenyl)-1-methylethyl]amino}ethyl)-2,5-dimethylphenyloxy]acetic acid,
and pharmacologically acceptable salts, enantiomers, diastereomers, tautomers, and metabolites thereof, and mixtures thereof.

5. Pharmaceutical composition according to one of claim 1, wherein:
the first active agent is selected from the group consisting of: tamsulosin, tamsulosin hydrochloride, finasteride, dutasteride, and pharmacologically acceptable salts, enantiomers, diastereomers, tautomers, and metabolites thereof, and mixtures thereof, and
the second active agent is selected from the group consisting of: (−)-ethyl-2-[4-(2-{[(1S,2R)-2-hydroxy-2-(4-hydroxyphenyl)-1-methylethyl]amino}ethyl)-2,5-dimethylphenyloxy]acetate, (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride, (−)-2-[4-(2-{[(1S,2R)-2-hydroxy-2-(4-hydroxyphenyl)-1-methylethyl]amino}ethyl)-2,5-dimethylphenyloxy]acetic acid, and pharmacologically acceptable salts, enantiomers, diastereomers, tautomers, and metabolites thereof, and mixtures thereof.

6. Pharmaceutical composition according to claim 1, comprising about 0.01 mg to about 5 mg of the first active agent, and about 10 mg to about 750 mg of the second active agent.

7. Pharmaceutical composition according to any one of claim 1, wherein the first and second active agents are formulated in the same pharmaceutical form.

8. Pharmaceutical composition according to any one of claim 1, wherein the first and second active agents are formulated in different pharmaceutical forms.

9. Pharmaceutical composition according to claim 1 adapted for rectal, topical, oral, sublingual, intranasal, transdermal, or parenteral administration.

10. Pharmaceutical composition according to claim 1 adapted for the simultaneous administration of the the first and second active agents.

11. Pharmaceutical composition according to claim 1, wherein the release of at least one of the first and second active agents is at least partially delayed after administration.

12. Pharmaceutical composition according to claim 1, wherein at least one of the first and second active agents is at least partially released immediately upon administration.

13. Pharmaceutical composition according to claim 1, comprising an alpha antagonist, a 5-alpha reductase inhibitor, and a beta-3-adrenoceptor agonist.

14. Method of treating a morbid change to or an irritation of the prostate in a mammal comprising administering to the mammal a pharmaceutical composition comprising: (a) a first active agent comprising a pharmaceutically effective amount of one or more alpha antagonists or 5-alpha reductase inhibitors, or a pharmacologically acceptable salt, enantiomer, diastereomer, tautomer, or metabolite thereof, and (b) a second active agent comprising a pharmaceutically effective amount of one or more beta-3-adrenoceptor agonists or a pharmacologically acceptable salt, enantiomer, diastereomer, tautomer, or metabolite thereof.

15. Method according to claim 14, wherein the first active agent is selected from the group consisting of: tamsulosin, tamsulosin hydrochloride, alfuzosin, bunazosin, doxazosin, indoramin, naftopidil, prazosin, terazosin, urapidil, silodosin, moxisylyte, metazosin, fiduxosin, upidosin, methyl 5-(N-(3-(4,4-diphenylpiperidin-1-yl)propyl)carbamoyl)-2,6-dimethyl-4(R)-(4-nitrophenyl)-1,4-dihydropyridin-3-carboxylate, AIO-8507L, 2-(3-(4-(5-chloro-2-methoxyphenyl)piperazin-1-yl)propylamino)pyrimidin-4-carboxamide fumarate), 5-methyl-3-(3-(4-(2-(2,2,2-trifluoroethoxy)phenyl)piperazin-1-yl)propyl)pyrimidin-2,4(1H,3H)-dione hydrochloride, and pharmacologically acceptable salts, enantiomers, diastereomers, tautomers, and metabolites thereof, and mixtures thereof.

16. Method according to claim 14, wherein the first active agent is selected from the group consisting of: tamsulosin, tamsulosin hydrochloride, finasteride, dutasteride, and pharmacologically acceptable salts, enantiomers, diastereomers, tautomers, and metabolites thereof, and mixtures thereof.

17. Method according to one of claim 14, wherein the second active agent is selected from the group consisting of:
(−)-ethyl-2-[4-(2-{[(1S,2R)-2-hydroxy-2-(4-hydroxyphenyl)-1-methylethyl]-amino}-ethyl)-2,5-dimethylphenyloxy]acetate, (−)-ethyl-2-[4-(2-{[(1S,2R)-2-hydroxy-2-(4-hydroxyphenyl)-1-methylethyl]-amino}-ethyl)-2,5-dimethylphenyloxy]acetate-monohydrochloride, and
(−)-2-[4-(2-{[(1S,2R)-2-hydroxy-2-(4-hydroxyphenyl)-1-methylethyl]amino}ethyl)-2,5-dimethylphenyloxy]acetic acid,
and pharmacologically acceptable salts, enantiomers, diastereomers, tautomers, and metabolites thereof, and mixtures thereof.

18. Method according to one of claim 14, wherein:
the first active agent is selected from the group consisting of: tamsulosin, tamsulosin hydrochloride, finasteride, dutasteride, and pharmacologically acceptable salts, enantiomers, diastereomers, tautomers, and metabolites thereof, and mixtures thereof, and
the second active agent is selected from the group consisting of: (−)-ethyl-2-[4-(2-{[(1S,2R)-2-hydroxy-2-(4-hydroxyphenyl)-1-methylethyl]amino}ethyl)-2,5-dimethylphenyloxy]acetate, (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride, (−)-2-[4-(2-{[(1S,2R)-2-hydroxy-2-(4-hydroxyphenyl)-1-methylethyl]amino}ethyl)-2,5-dimethylphenyloxy]acetic acid, and pharmacologically acceptable salts, enantiomers, diastereomers, tautomers, and metabolites thereof, and mixtures thereof.

19. Method according to claim 14, comprising about 0.01 mg to about 5 mg of the first active agent, and about 10 mg to about 750 mg of component the second active agent.

20. Method according to one of claim 14, wherein the pharmaceutical composition comprises an alpha antagonist, a 5-alpha reductase inhibitor, and a beta-3-adrenoceptor agonist.
